(19) United States
(12) Patent Application Publication
(10) Pub. No.: US 2021/0249109 A1
(43) Pub. Date: Aug. 12, 2021

(54) INFORMATION PROCESSING SYSTEM, INFORMATION PROCESSING DEVICE, AND INFORMATION PROCESSING METHOD

(71) Applicant: Sony Corporation, Tokyo (JP)

(72) Inventors: Shunsuke HAYASHI, Tokyo (JP); Hideto SHIKATA, Tokyo (JP); Hiroo MIURA, Tokyo (JP); Akihiko NAKATANI, Tokyo (JP); Koji OBATA, Tokyo (JP)

(73) Assignee: Sony Corporation, Tokyo (JP)

(21) Appl. No.: 16/973,436

(22) PCT Filed: Jun. 18, 2019
(86) PCT No.: PCT/JP2019/024149

§ 371 Date: Dec. 9, 2020

(30) Foreign Application Priority Data
Jun. 18, 2018 (JP) 2018-115182

(51) Int. Cl. G16H 10/60; G06F 21/62

(52) U.S. Cl. CPC G16H 10/60; G06F 21/6245

(57) ABSTRACT

An information processing system includes an acquisition unit configured to acquire a first surgical record that is a record of information regarding an operation of a medical device used in surgery, a conversion unit (101) configured to perform protection processing for personal information of a patient included in the first surgical record to convert the first surgical record into a second surgical record, an identification information generation unit (102) configured to generate first identification information associated with the first surgical record and second identification information associated with the second surgical record, and a determination unit (103) configured to determine which of the first surgical record associated with the first identification information and the second surgical record associated with the second identification information is to be transmitted to a node (30) used by the user on the basis of authority information regarding an authority of the user.

### TECHNICAL FIELD

The present disclosure relates to an information processing system, an information processing device, and an information processing method.

### BACKGROUND ART

In recent years, in a medical setting, recording of images obtained with an endoscope during surgery, images obtained with an operative field camera, operating conditions of an electric scalpel and the like, and the like has been performed in order to analyze a surgical state after surgery. Furthermore, these surgical records may be used for publication for academic conferences or training purposes, or for storage for use in statistical analysis, for example.

However, since the surgical record may include personal information such as the patient's face and name, protection of the personal information is required when the surgical record is shared by a large number of people. For example, Patent Document 1 below discloses a technique of deleting personal information from a moving image file including a medical image and the personal information without manual work.

### CITATION LIST

### Patent Document

### Patent Document 1: Japanese Patent Application Laid-Open No. 2012-217631

### SUMMARY OF THE INVENTION

### Problems to be Solved by the Invention

However, according to the technique disclosed in Patent Document 1, in a case where use restriction is not placed on the moving image file from which the personal information has not been deleted, a user can use the moving image file. As a result, there is a possibility that a moving image file from which the personal information has not been deleted is acquired by an unspecified person, and the personal information of the patient is leaked. Thus, from the viewpoint of sharing surgical records, protection of personal information is required, while from the viewpoint of recording surgical conditions, storage of unaltered surgical records is required.

Therefore, the present disclosure proposes an information processing system, an information processing device, and an information processing method capable of achieving both storage of surgical records and protection of personal information.

### Solutions to Problems

According to the present disclosure, there is provided an information processing system including an acquisition unit configured to acquire a first surgical record that is a record of information regarding an operation of a medical device used in surgery in a hospital, a conversion unit configured to perform protection processing for personal information of a patient included in the first surgical record to convert the first surgical record into a second surgical record, an identification information generation unit configured to generate first identification information associated with the first surgical record and second identification information associated with the second surgical record, and a determination unit configured to determine whether or not the user is accessible to either the first surgical record associated with the first identification information or the second surgical record associated with the second identification information on the basis of authority information regarding an authority of the user determined on the basis of an attribute in the hospital or information of the surgery.

Furthermore, according to the present disclosure, there is provided an information processing device including a determination unit configured to determine which of a first surgical record associated with first identification information and a second surgical record associated with second identification information is to be transmitted to a node used by a user on the basis of authority information regarding an authority of the user, in which the first surgical record is a record of information regarding an operation of a medical device used in surgery, and the second surgical record is a record converted from the first surgical record when protection processing is performed for personal information of a patient included in the first surgical record.

Furthermore, according to the present disclosure, there is provided an information processing method including, by a processor, acquiring a first surgical record that is a record of information regarding an operation of a medical device used in surgery, performing protection processing for personal information of a patient included in the first surgical record to convert the first surgical record into a second surgical record that is a surgical record from which the patient is unspecifiable, generating first identification information associated with the first surgical record and second identification information associated with the second surgical record, and determining which of the first surgical record associated with the first identification information and the second surgical record associated with the second identification information is to be transmitted to a node used by a user on the basis of authority information regarding an authority of the user.

According to the present disclosure, whether or not to transmit a moving image for which protection processing for personal information has been performed to a node used by a user is determined according to user's authority.

### Effect of the Invention

As described above, according to the present disclosure, both the storage of surgical records and the protection of personal information can be achieved.

Note that the above-described effect is not necessarily restrictive, and any one of effects described in the present specification or any another effect obtainable from the present specification may be exhibited in addition to or in place of the above-described effect.

### BRIEF DESCRIPTION OF DRAWINGS

FIG. 1 is an explanatory diagram illustrating a configuration of an information processing system according to a first embodiment of the present disclosure.

FIG. 2 is a block diagram illustrating a configuration of an information processing device according to the embodiment.

FIG. 3 is a diagram illustrating an example of an authority information list used for determination by a determination unit according to the embodiment.

FIG. 4 is an explanatory diagram illustrating a configuration of a surgical system according to the embodiment.

FIG. 5 is a flowchart illustrating an operation flow from acquisition to storage of a first surgical moving image by the information processing device according to the embodiment.

FIG. 6 is a flowchart illustrating an operation flow of the determination by the determination unit according to the embodiment.

FIG. 7 is an explanatory diagram illustrating a configuration of an information processing device according to a second embodiment of the present disclosure.

FIG. 8 is a diagram illustrating an example of an authority information list according to the embodiment.

FIG. 9 is a flowchart illustrating an operation flow from acquisition to storage of a first surgical moving image by the information processing device according to the embodiment.

FIG. 10 is a flowchart illustrating an operation flow of the determination by a determination unit according to the embodiment.

FIG. 11 is an explanatory diagram illustrating display output to a node according to the embodiment.

FIG. 12 is an explanatory diagram illustrating another display output to a node according to the embodiment.

FIG. 13 is a block diagram illustrating a configuration example of an information processing system according to a third embodiment of the present disclosure.

FIG. 14 is a sequence diagram illustrating an operation flow by the information processing system according to the embodiment.

FIG. 15 is a sequence diagram illustrating an operation flow from acquisition to storage of a second surgical moving image in a case where a first surgical moving image and the second surgical moving image are respectively stored in different storage servers, by the information processing system according to the embodiment.

FIG. 16 is a block diagram illustrating a hardware configuration of the information processing system according to the present disclosure.

FIG. 17 is a block diagram illustrating a modification of the configuration of the information processing system according to the present disclosure.

### MODE FOR CARRYING OUT THE INVENTION

Favorable embodiments of the present disclosure will be described in detail with reference to the appended drawings. Note that, in the present specification and drawings, redundant description of configuration elements having substantially the same functional configuration is omitted by providing the same sign.

Note that the description will be given in the following order.

<1. First Embodiment>

<2. Second Embodiment>

<3. Third Embodiment>

<4. Hardware Configuration>

<5. Conclusion>

### 1. First Embodiment

[1.1. Configuration]

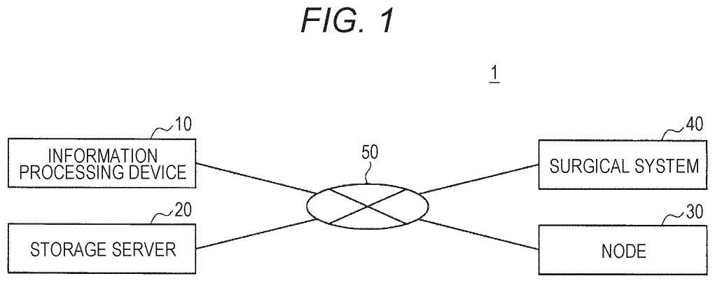

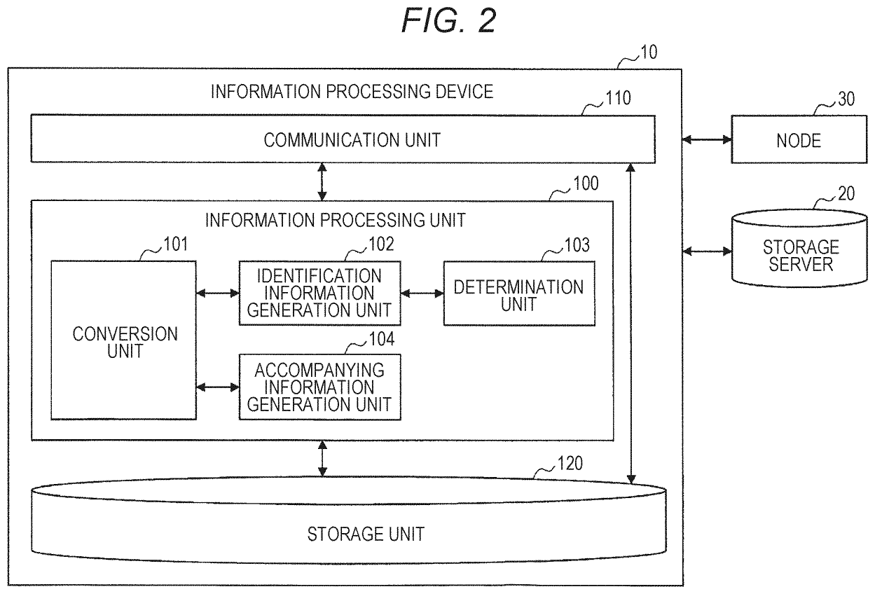

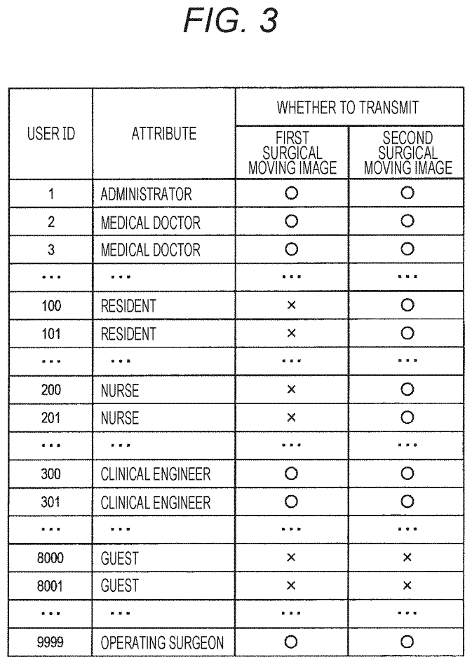

First, an information processing system 1 according to a first embodiment of the present disclosure will be described with reference to FIGS. 1 to 3. FIG. 1 is an explanatory diagram illustrating the information processing system 1 according to the present embodiment. FIG. 2 is a block diagram illustrating a configuration of an information processing device 10 according to the present embodiment. FIG. 3 is a diagram illustrating an example of an authority information list used for determination by a determination unit 103 according to the present embodiment.

The information processing system 1 according to the present embodiment includes the information processing device 10, a storage server 20, a node 30, a surgical system 40, and a network 50.

The information processing system 1 has functions to acquire a first surgical record that is a record of information regarding an operation of a medical device used in surgery, perform protection processing for personal information of a patient included in the first surgical record, and convert the first surgical record into a second surgical record. The information processing system 1 generates first identification information associated with the first surgical record and second identification information associated with the second surgical record. The information processing system 1 determines which of the first surgical record associated with the first identification information and the second surgical record associated with the second identification information is to be transmitted to a node used by a user on the basis of authority information regarding an authority of the user. In the present specification, the present technology will be described using a case as an example, in which the first surgical record is a first surgical moving image that is a record of information regarding an operation of a medical device used in surgery by a moving image, and the second surgical record is a second surgical moving image converted from the first surgical moving image when the protection processing is performed for the personal information of the patient included in the first surgical moving image.

Note that the first surgical moving image includes all moving images acquirable during surgery. The first surgical moving image includes, for example, a moving image acquired by an endoscope, a moving image acquired by an operative field camera installed in an operating room, a moving image of a display screen of a peripheral measuring instrument, a moving image that captures an operating state of a device used for surgery, and the like. Furthermore, the first surgical moving image may include a moving image acquired by a camera that captures a state in a line-of-sight direction of a person involved in surgery, for example, an operating surgeon, a nurse, a clinical engineer, or the like. The camera that captures a state in the line-of-sight of a person involved in surgery is attached to, for example, a glasses-type wearable device or a head mounted display (HMD). Furthermore, the first surgical moving image may include a moving image acquired by a surgical robot. Note that the first surgical moving image may include a plurality of moving images acquirable during surgery.

Furthermore, the personal information referred to here can identify a specific individual. The personal information includes information that can identify a specific individual by collating the information with another information. The personal information includes, for example, the face and name of the patient recorded in the moving image, the name and voice of the patient included in sound in the moving image, and the like. Furthermore, the personal information can include not only the entire face but also skeleton and skin color of the face, appearance determined by the position and shape of eyes, nose, mouth and other parts of the face, and a linear shape formed by undulations of a surface of an iris. Furthermore, the personal information may include, for example, a voiceprint of the patient as a characteristic of the voice of the patient. Moreover, the personal information may include the shape of veins, fingerprints, or palm prints defined by branches and end points of subcutaneous veins of the patient's palm, back of the hand, or fingers. Furthermore, the personal information may be one that can identify a specific individual by being combined with a plurality of pieces of information. For example, information such as patient's age, number of days to discharge from hospital, medical history, or the like, which cannot specify individual but becomes able to specify individual in combination with a plurality of pieces of information, may be treated as the personal information.

The protection processing for the personal information is favorably performed by a method that can prevent the patient from being irreversibly identified. For example, the protection processing is desirably performed by mosaic processing or black painting processing for the personal information. The protection of the personal information may be performed by advanced encryption processing that cannot be substantially decrypted without an encryption key. In the protection processing for the personal information, whether or not the surgical moving image includes the face of the patient or the face of an operating room member is determined by face recognition using machine learning, for example. Then, the mosaic processing or the black painting processing is performed for an area recognized as a face. Note that, in the face recognition using machine learning, an image associated with a label indicating an area including a human face is input as learning data to a machine learning model of a multilayer neural network and parameters are generated, for example. A predetermined one frame of the surgical moving image is input to an inference model of the multilayer neural network set on the basis of the parameters. Thereby, an area including the face can be estimated. Furthermore, the protection of the personal information may be performed by image processing of replacing the face or voice of the patient with another face or voice. Thereby, a facial expression of the patient during surgery can be observed while protecting the personal information by the replacement processing although the facial expression of the patient cannot be observed in the mosaic processing of the black painting processing. For example, a face area of the patient is determined by the face recognition using machine learning. Next, the replacement processing of replacing the patient's face with another human face is performed. By performing the face replacement processing for each frame of the surgical moving image, the personal information is protected. Furthermore, a facial expression of the patient (a change in the patient's face during surgery) can be recorded in the second surgical moving image. Note that the face and voice replacement processing is favorably performed by reinforcement learning (for example, generative adversarial networks) using a multilayer neural network. Furthermore, a mark indicating whether or not the face has been replaced may be superimposed on the second surgical moving image. Thereby, in a case where a face supposed to be the patient's face is included in the second surgical moving image, determination as to whether or not the personal information has been protected can be easily performed.

As illustrated in FIG. 2, the information processing device 10 includes an information processing unit 100, a communication unit 110, and a storage unit 120. The information processing unit 100 has a function to control processing for a surgical moving image acquired by the information processing device 10. The information processing unit 100 includes a conversion unit 101, an identification information generation unit 102, the determination unit 103, and an accompanying information generation unit 104.

The conversion unit 101 has functions of an acquisition unit that acquires the first surgical moving image, and a conversion unit that performs the protection processing for personal information of a patient included in the first surgical moving image to convert the first surgical moving image into a second surgical moving image. The conversion unit 101 has a function to acquire the first surgical moving image from the surgical system 40 via the communication unit 110, and perform the protection processing for the personal information to convert the first surgical moving image into the second surgical moving image in which the patient cannot be specified in the case where the personal information of the patient is included in the acquired first surgical moving image. Specifically, the conversion unit 101 detects the personal information in the first surgical moving image. In the case where the personal information has been detected from the first surgical moving image, the conversion unit 101 determines a personal information area in the first surgical moving image and performs the protection processing for the personal information area. The detection of the personal information and the determination of the personal information area can be performed using existing image recognition technology and image processing technology. The protection processing for the personal information with the determined area can be performed by a method for preventing the patient from being irreversibly specified, as described above. Furthermore, the first surgical moving image before being converted into the second surgical moving image is stored in the storage server 20 or the storage unit 120. Note that, hereinafter, in a case where there is no need to distinguish the first surgical moving image and the second surgical moving image, the first surgical moving image and the second surgical moving image may be simply referred to as surgical moving images.

In a case where the personal information is included as sound data in the first surgical moving image, the conversion unit 101 can process the sound data including information corresponding to the personal information into sound data not corresponding to the personal information by an existing sound processing technology. For example, the conversion unit 101 may convert the sound data of a portion corresponding to the personal information into sound data not corresponding to the personal information, may delete the sound data of a portion corresponding to the personal information, or the like.

Regarding the presence or absence of the personal information in the first surgical moving image, the personal information in surgery is often included in a moving image acquired by an operative field camera. Therefore, in a case where it is clear that the personal information is not included in another moving image included in the first surgical moving image, the conversion unit 101 can determine the presence or absence of the personal information in the first surgical moving image according to whether or not an image of the operative field camera is included in the first surgical moving image, for example. For example, in a case where the first surgical moving image is determined to be an endoscopic moving image on the basis of accompanying information (for example, metadata) of the moving image, the possibility that the personal information is captured is very low. Therefore, it may be determined that the personal information is not included in the first surgical moving image.

Note that the second surgical moving image may include a surgical moving image converted into a moving image for which the protection processing for the personal information has been performed, of the first surgical moving image, and a surgical moving image for which the protection processing for the personal information has not been performed, of the first surgical moving image. Furthermore, the second surgical moving image may include only the surgical moving image converted into a moving image for which the protection processing for the personal information has been performed, of the first surgical moving image.

The identification information generation unit 102 has a function to generate identification information for identifying the first surgical moving image and the second surgical moving image. Specifically, the identification information generation unit 102 sets a corresponding flag for each of the first surgical moving image and the second surgical moving image, and recognizes the flags as the identification information. The identification information includes the first identification information associated with the first surgical moving image and the second identification information associated with the second surgical moving image. Hereinafter, there is no need to specifically distinguish the first identification information and the second identification information, the first identification information and the second identification information are simply referred to as identification information, and the identification information associated with the first surgical moving image is referred to as the first identification information, and the identification information associated with the second surgical moving image is referred to as the second identification information, as needed. The first identification information is identification information indicating that the first surgical moving image includes the personal information, and is metadata indicating that the personal information is included, for example. The second identification information is identification information indicating that the second surgical moving image does not include the personal information, and is metadata indicating that the personal information is not included, for example. Note that it is sufficient that the first identification information and the second identification information can be determined to include or not to include personal information, and may directly include the presence or absence of personal information in the data or indirectly include the presence or absence of personal information in the data. For example, the presence or absence of the personal information may be directly included in the data by a flag indicating the presence or absence of the personal information. Furthermore, for example, the presence or absence of the personal information may be indirectly included in the data by information indicating a user who can access the first identification information or the second identification information.

The determination unit 103 has a function to determine which of the first surgical record associated with the first identification information and the second surgical record associated with the second identification information is to be transmitted to the node 30 used by a user on the basis of authority information regarding an authority of the user. The authority information includes information regarding the user, information regarding an access authority to the surgical moving image, and the like. For example, the authority information includes attributes, information regarding whether to transmit the first surgical moving image and the second surgical moving image, and the like, which are associated with a user ID, as illustrated in FIG. 3. For example, a user ID “1” is associated with information that the attribute is an administrator, and both the first surgical moving image and the second surgical moving image can be transmitted to the node 30 that has transmitted the user ID “1”. Similarly, for example, a user ID “2” is associated with information that the attribute is a medical doctor, and both the first surgical moving image and the second surgical moving image can be transmitted to the node 30 that has transmitted the user ID “2”. Furthermore, for example, a user ID “100” is associated with information that the attribute is a resident, and the first surgical moving image cannot be transmitted but the second surgical moving image can be transmitted to the node 30 that has transmitted the user ID “100”. Furthermore, for example, a user ID “8000” is associated with information that the attribute is a guest, and both the first surgical moving image and the second surgical moving image cannot be transmitted to the node 30 that has transmitted the user ID “8000”. Note that the authority information is favorably information based on an attribute in the hospital. For example, the authority information may be determined on the basis of an attribute (for example, a medical doctor, a resident, a nurse, or the like) related to a function included in a personnel information database in the hospital. Furthermore, the authority information may be determined on the basis of information of a member related to surgery included in a surgical information database that records information regarding surgery or information indicating the function (for example, an operating surgeon, an assistant, a nurse, or the like) in the surgery.

The accompanying information generation unit 104 has a function to generate accompanying information accompanying the second surgical moving image and related to the personal information. The accompanying information includes, for example, information regarding the personal information included in the second surgical moving image, and specifically includes information of timing at which or time for which the personal information is included in the moving image. The accompanying information generation unit 104 can generate the accompanying information using an existing image processing technology. The accompanying information generation unit 104 may generate accompanying information accompanying the first surgical moving image. Hereinafter, the accompanying information accompanying the first surgical moving image may be referred to as first accompanying information, and the accompanying information accompanying the second surgical moving image may be referred to as second accompanying information, as needed.

The storage unit 120 appropriately stores various programs, databases, and the like used when the information processing device 10 executes the above-described various types of processing. Furthermore, the storage unit 120 may record various types of information generated by the information processing unit 100 as history information. The storage unit 120 may store, for example, the first surgical moving image, the second surgical moving image, the identification information, the authority information, the accompanying information, and the like. Moreover, the storage unit 120 may appropriately store various parameters, progresses of the processing, and the like that need to be saved when the conversion unit 101, the identification information generation unit 102, the determination unit 103, and the accompanying information generation unit 104 perform the above-described processing, for example. The storage unit 120 may appropriately store various parameters, progresses of the processing, and the like that need to be saved when not only the conversion unit 101, the identification information generation unit 102, the determination unit 103, and the accompanying information generation unit 104 but also the information processing device 10 according to the present embodiment performs some processing. The information processing unit 100 and the communication unit 110 can freely perform read/write processing on the storage unit 120.

The communication unit 110 has a function to transmit and receive various types of information to and from the surgical system 40, the storage server 20, and the node 30 via the network 50. Specifically, the communication unit 110 receives the first surgical moving image from the surgical system 40 and outputs the first surgical moving image to the information processing unit 100. Furthermore, the communication unit 110 transmits the first surgical moving image, the second surgical moving image converted from the first surgical moving image, the various types of information generated in the information processing unit 100, such as the identification information and the accompanying information, the authority information received from the node 30, and the like to the storage server 20.

The storage server 20 stores the first surgical moving image, the second surgical moving image, the identification information, the authority information, and the accompanying information. The storage server 20 is connected to the information processing device 10, the node 30, and the surgical system 40 via the network 50. The storage server 20 stores the first surgical moving image, the second surgical moving image, the identification information, the authority information, and the accompanying information received from the information processing device 10, but the storage server 20 may store the authority information received from the node 30 or may store the first surgical moving image received from the surgical system 40.

Furthermore, the first surgical moving image, the second surgical moving image, the identification information, and the accompanying information may be stored in a folder to which a unique surgery ID is assigned for each surgery, and the folder may be stored in the storage server 20 or the storage unit 120. A number or a symbol is assigned to the surgery ID assigned at this time, for each type of information regarding the surgery, such as the type of the surgery, a surgical site, an operating surgeon, or date. The surgery ID may be assigned by a combination of these numbers or symbols. The surgical moving image, the identification information, and the accompanying information regarding predetermined surgery become able to be transmitted to the node 30 on the basis of the surgery ID.

The node 30 is used when accessing the surgical moving image, and is connected to the information processing device 10, the storage server 20, and the surgical system 40 by the network 50. The node 30 has a display for displaying at least one of the first surgical moving image or the second surgical moving image. Each user who uses the node 30 is assigned a unique user ID owned by the each user. When the user uses the node 30, for example, the user ID is transmitted from the node 30 to the information processing device 10 when the user logs in to the node 30. The transmitted user ID is used for the determination by the determination unit 103. Note that the plurality of nodes 30 may be connected to the information processing device 10 and the storage server 20 via the network 50.

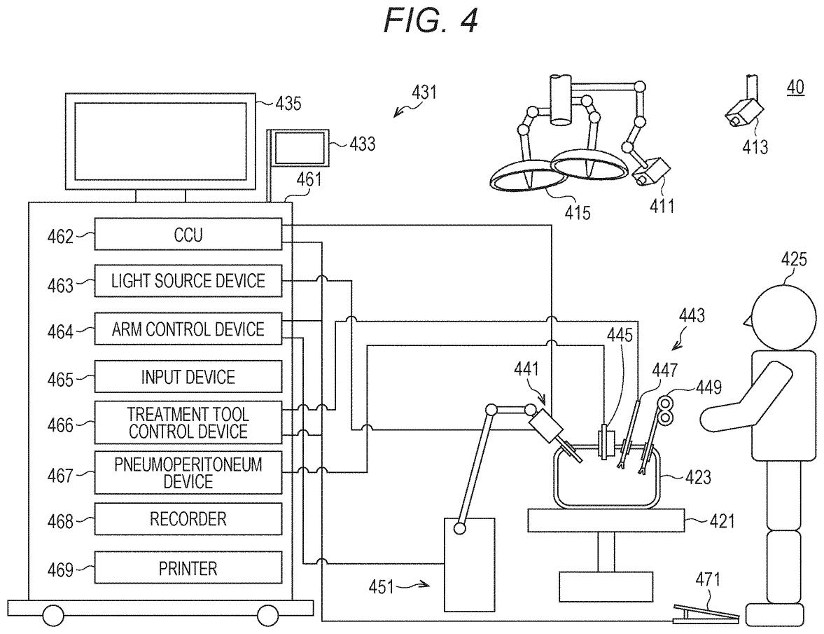

The surgical system 40 has a function to acquire the first surgical moving image. Here, an example of the surgical system 40 will be described with reference to FIG. 4. FIG. 4 is an explanatory diagram illustrating a configuration of the surgical system 40 according to the present embodiment.

In an operating room to which the surgical system 40 is applied, a ceiling camera 411 and a surgical field camera 413 are provided on a ceiling of the operating room, for example. The ceiling camera 411 and the surgical field camera 413 can image hands of an operator (surgeon) 425 who performs treatment for an affected part of a patient 423 on a patient bed 421 and a state of the entire operating room. The ceiling camera 411 and the surgical field camera 413 can be provided with a magnification adjustment function, a focal length adjustment function, an imaging direction adjustment function, and the like. An illumination 415 is provided on the ceiling of the operating room and illuminates at least the hands of the operator 425. The illumination 415 may be able to appropriately adjust an irradiation light amount, a wavelength (color) of irradiation light, an irradiation direction of the light, and the like.

An endoscopic surgical system 431, the patient bed 421, the ceiling camera 411, the surgical field camera 413, and the illumination 415 are connected in cooperation with one another. A centralized operation panel 433 is provided in the operating room, and the user can appropriately operate these devices present in the operating room via the centralized operation panel 433, as described above.

Hereinafter, a configuration that can be adopted by the surgical system 40 will be described by taking an endoscopic surgery as an example. As illustrated in FIG. 4, the surgical system 40 may include an endoscope 441, surgical tools 443, a support arm device 451 that supports the endoscope 441, and a cart 461 on which various devices for endoscopic surgery are mounted.

In the endoscopic surgery, a plurality of cylindrical puncture devices is punctured into an abdominal wall instead of cutting the abdominal wall to open the abdomen. The endoscope 441 and the surgical tools 443 are inserted into the body cavity of the patient 423 through the opening formed by the puncture devices. In the illustrated example, as the surgical tools 443, a pneumoperitoneum tube 445, an energy treatment tool 447, and a pair of forceps 449 are inserted into the body cavity of the patient 423. The energy treatment tool 447 is a treatment tool for performing incision and detachment of tissue, sealing of a blood vessel, and the like with a high-frequency current or an ultrasonic vibration.

An image of an operation site in the body cavity of the patient 423 captured by the endoscope 441 is displayed on a display device 435. The operator 425 performs treatment such as removal of an affected part, using the energy treatment tool 447 and the pair of forceps 449 while viewing the image of the operation site displayed on the display device 435 in real time.

The support arm device 451 supports the endoscope 441 and controls the position and posture of the endoscope 441. With the control, stable fixation of the position of the endoscope 441 can be realized.

An area of a predetermined length from a tip of the endoscope 441 is inserted into the body cavity of the patient 423. A light source device 463 is connected to the endoscope 441, and light generated by the light source device 463 is emitted to an observation target inside the body cavity of the patient 423.

A camera control unit (CCU) 462 includes a central processing unit (CPU), a graphics processing unit (GPU), and the like, and centrally controls the operations of the endoscope 441 and the display device 435. Specifically, the CCU 462 provides an image signal to the display device 435. Furthermore, the CCU 462 transmits a control signal to the endoscope 441 to control its driving. The control signal may include information regarding imaging conditions such as a magnification and a focal length. The information regarding imaging conditions may be input via an input device 465 or may be input via the above-described centralized operation panel 433.

The display device 435 displays an image based on the image signal under the control of the CCU 462. Furthermore, a plurality of the display devices 435 having different resolutions and sizes may be provided depending on the use.

The light source device 463 includes a light source such as a light emitting diode (LED), for example, and supplies irradiation light to the endoscope 441 in imaging an operation site.

An arm control device 464 includes a processor such as a CPU, and operates according to a predetermined program, thereby controlling driving of an arm unit of the support arm device 451 according to a predetermined control method.

The input device 465 is an input interface for the endoscopic surgical system 431. The user can input various types of information and instructions to the endoscopic surgical system 431 via the input device 465. For example, the user inputs various types of information regarding surgery, such as the patient's physical information and information regarding an operation method of the surgery via the input device 465. Furthermore, for example, the user inputs an instruction to drive the arm unit, an instruction to change the imaging conditions (such as the type of the irradiation light, the magnification, and the focal length) of the endoscope 441, an instruction to drive the energy treatment tool 447, or the like via the input device 465.

The type of the input device 465 is not limited, and the input device 465 may be one of various known input devices. For example, a mouse, a keyboard, a touch panel, a switch, a foot switch 471, a lever, and/or the like can be applied to the input device 465. In the case where a touch panel is used as the input device 465, the touch panel may be provided on a display surface of the display device 435.

Alternatively, the input device 465 is, for example, a device worn by the user, such as a glass-type wearable device or a head mounted display (HMD), and various inputs are performed according to a gesture or a line-of-sight of the user detected by the device. Furthermore, the input device 465 includes a camera capable of detecting a movement of the user, and various inputs are performed according to a gesture or a line-of-sight of the user detected from an image imaged by the camera. Moreover, the input device 465 includes a microphone capable of collecting a voice of the user, and various inputs are performed by a sound through the microphone. The input device 465 is thus configured to be able to input various types of information in a non-contact manner, so that the user (for example, the operator 425) in particular belonging to a clean area can operate a device belonging to a filthy area in a non-contact manner.

A treatment tool control device 466 controls driving of the energy treatment tool 447 for cauterization and incision of tissue, sealing of a blood vessel, and the like. A pneumoperitoneum device 467 sends a gas into the body cavity of the patient 423 through the pneumoperitoneum tube 445 to expand the body cavity for the purpose of securing a field of vision by the endoscope 441 and a work space for the operator. A recorder 468 is a device capable of recording various types of information regarding surgery. A printer 469 is a device capable of printing the various types of information regarding surgery in various formats such as a text, an image, or a graph.

The above-described surgical system 40 is merely an example, and devices and equipment other than those described above may be applied or the above-described devices and equipment may be omitted as necessary.

The surgical system 40 may include a display screen of a peripheral measuring instrument and a camera capable of acquiring a moving image of an operation state of the device. The first surgical moving image is acquired by the various cameras provided in the above-described surgical system 40.

[1.2. Operation]

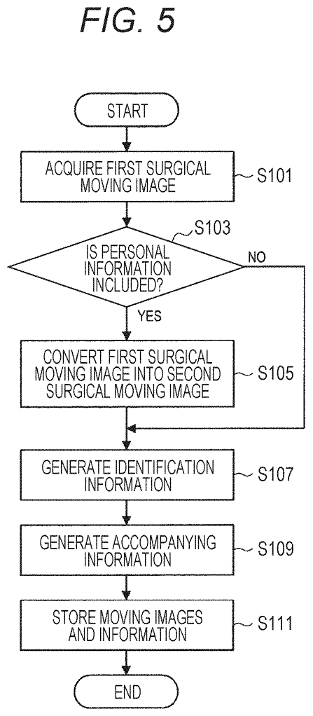

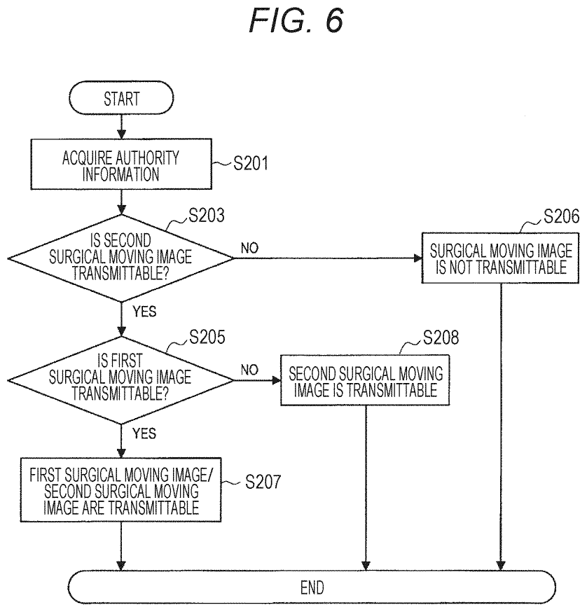

A configuration of the information processing system 1 according to the present embodiment has been described. Next, an operation of the information processing device 10 will be described with reference to FIGS. 5 and 6. FIG. 5 is a flowchart illustrating an operation flow from acquisition to storage of the first surgical moving image by the information processing device 10 according to the present embodiment. FIG. 6 is a flowchart illustrating an operation flow of determination by the determination unit 103 according to the present embodiment.

An operation flow from acquisition of the first surgical moving image to storage of the first surgical moving image, the second surgical moving image, and the accompanying information by the information processing device 10 will be described with reference to FIG. 5.

First, the information processing device 10 acquires the first surgical moving image from the surgical system 40 provided in the operating room (S101). Specifically, the communication unit 110 receives the first surgical moving image from the surgical system 40 via the network 50 and outputs the first surgical moving image to the information processing unit 100. At this time, the first surgical moving image may be stored in the storage server 20 or the storage unit 120.

Next, the conversion unit 101 included in the information processing unit 100 determines whether or not the first surgical moving image includes the personal information (S103). In a case where the personal information is included in the first surgical moving image (S103/YES), the first surgical moving image is converted into the second surgical moving image (S105). Next, the identification information generation unit 102 generates the identification information corresponding to the first surgical moving image and the second surgical moving image (S107). Specifically, the identification information generation unit 102 sets flags respectively corresponding to the first surgical moving image and the second surgical moving image to generate the identification information to be associated with the first surgical moving image and the second surgical moving image.

Next, the accompanying information generation unit 104 generates the accompanying information accompanying the second surgical moving image (S109). Specifically, the accompanying information generation unit 104 can generate information of timing at which or time for which the personal information is included in the moving image, using an existing image processing technology.

The first surgical moving image, the second surgical moving image, the identification information, and the accompanying information are transmitted to and stored in the storage server 20 via the network 50 (S111). Note that the first surgical moving image, the second surgical moving image, the identification information, and the accompanying information may be stored in the storage unit 120.

Meanwhile, in S103, in a case where the personal information is not included in the first surgical moving image (S103/NO), the above-described conversion processing into the second surgical moving image in S105 is not executed, and the identification information generation unit 102 generates the identification information corresponding to the first surgical moving image (S107).

Furthermore, generation of the identification information corresponding to the first surgical moving image may be executed before the conversion to the second surgical moving image (S105) or may be executed at the same time with the conversion to the second surgical moving image. Furthermore, the order of the operations of the identification information generation (S107) and the accompanying information generation (S109) is not limited to the above description, and the identification information may be generated after the accompanying information is generated.

The operation flow from acquisition to storage of the first surgical moving image by the information processing device 10 has been described. Next, an operation flow of the determination by the determination unit 103 will be described with reference to FIG. 6.

First, the determination unit 103 acquires the user ID from the node 30 (S201). At this time, the determination unit 103 receives the surgery ID input by the user from the node 30, thereby acquiring the first surgical moving image, the second surgical moving image, the first accompanying information, and the second accompanying information associated with the surgery ID from the storage server 20.

Next, the determination unit 103 determines whether to transmit the second surgical moving image to the node 30 on the basis of the authority information (S203). The determination unit 103 searches the authority information list illustrated in FIG. 3 for the user ID transmitted from the node 30, and determines whether to transmit the second surgical moving image to the node 30 according to the authority information associated with the user ID, for example. For example, as illustrated in FIG. 3, the determination unit 103 determines that the second surgical moving image can be transmitted to the node 30 in the case where the node 30 is used by the user having the user ID “1”.

Next, in the case where the determination unit 103 determines that the second surgical moving image can be transmitted to the node 30 (S203/YES), the determination unit 103 determines whether or not to transmit the first surgical moving image to the node 30 on the basis of the authority information (S205). For example, as illustrated in FIG. 3, the determination unit 103 determines that the first surgical moving image can be transmitted to the node 30 in the case where the determination unit 103 has received any of the user IDs “1” to “3”, “300”, “301”, and “9999” (S205/YES). As a result, the determination unit 103 determines that the first surgical moving image associated with the surgery ID and the second surgical moving image associated with the surgery ID can be transmitted to the node 30 (S207).

In S205, for example, as illustrated in FIG. 3, the determination unit 103 determines that the first surgical moving image cannot be transmitted to the node 30 in the case where the determination unit 103 has received any of the user IDs “100”, “101”, “200”, and “201” (S205/NO). As a result, the determination unit 103 determines that the second surgical moving image associated with the surgery ID can be transmitted to the node 30 (S208).

In S203, for example, as illustrated in FIG. 3, the determination unit 103 determines that the second surgical moving image cannot be transmitted to the node 30 in the case where the determination unit 103 has received the user ID “9999” (S205/NO). In this case, the determination unit 103 determines that neither the first surgical moving image nor the second surgical moving image can be transmitted to the node 30 (S206).

According to the present embodiment, the unmodified surgical moving image is stored, and whether to transmit the surgical moving image to the node used by the user is determined. Therefore, the unmodified surgical moving image including the personal information can be used only by an authorized user. As a result, leakage of the personal information is prevented. Therefore, the information processing system according to the present embodiment can achieve both the storage of the surgical record and the protection of the personal information.

[1.3. Modification]

In the above embodiment, a case in which one storage server 20 is provided has been described, but the information processing system according to the present embodiment may include a plurality of storage servers. For example, in a case where two storage servers are provided, one storage server may store the first surgical moving image and the other storage server may store the second surgical moving image. Since the first surgical moving image and the second surgical moving image are stored in different storage servers, security is enhanced and leakage of the personal information can be prevented.

### 2. Second Embodiment

[2.1. Configuration]

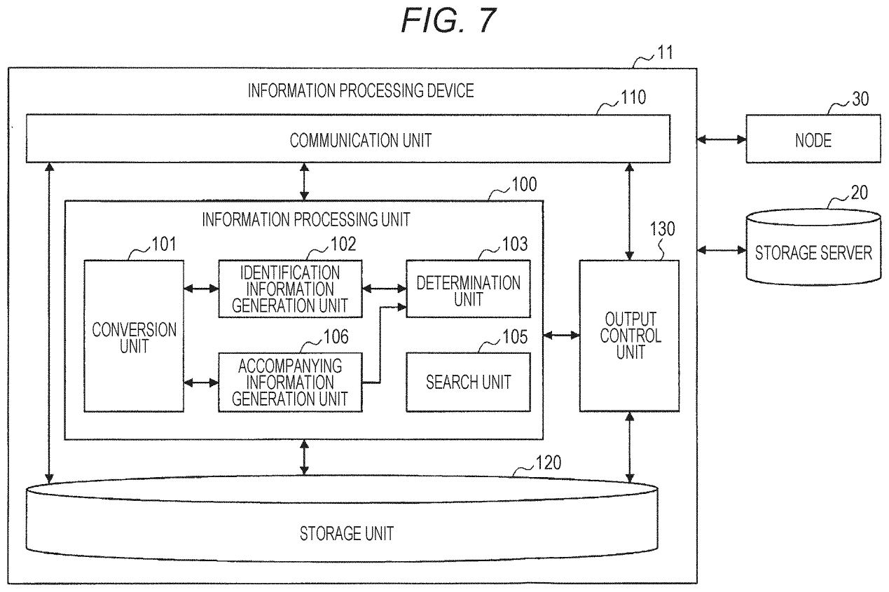

Next, an information processing system 2 according to the second embodiment of the present disclosure will be described with reference to FIG. 7. FIG. 7 is a block diagram illustrating a configuration example of the information processing system 2 according to the present embodiment. The information processing system 2 according to the present embodiment includes an information processing device 11, a storage server 20, a node 30, a surgical system 41, and a network 50. In the present embodiment, the storage server 20 and the node 30 applicable to the information processing system 2 are similar to those described in the first embodiment, and thus detailed description thereof is omitted here.

The information processing device 11 according to the present embodiment includes an information processing unit 100, a communication unit 110, a storage unit 120, and an output control unit 130. The information processing unit 100 includes a conversion unit 101, an identification information generation unit 102, a determination unit 103, a search unit 105, and an accompanying information generation unit 106. In the present embodiment, the conversion unit 101, the identification information generation unit 102, and the determination unit 103 are similar to those described in the first embodiment, and thus detailed description thereof is omitted here.

The accompanying information generation unit 106 has a function to generate accompanying information accompanying any information related to surgery in addition to information related to personal information. For example, the accompanying information generation unit 106 can generate, as the accompanying information, information such as a type of surgery, a flow of the surgery, a progress of the surgery, the amount of bleeding, a location of bleeding, occurrence timing of bleeding, and timing at which a medical device is used. The accompanying information generation unit 106 can generate, for example, chapter information indicating timing at which a predetermined operation has been performed as the accompanying information by applying a technology of adding information to a moving image file disclosed in Japanese Patent Application Laid-Open No. 2016-42982. Furthermore, the accompanying information generation unit 106 can generate, for example, occurrence timing of bleeding or timing at which a medical device is used as the accompanying information by applying an image recording technology disclosed in Japanese Patent Application Laid-Open No. 2011-36372. Furthermore, the accompanying information generation unit 106 can generate, for example, information regarding the location of bleeding and the amount of bleeding in a surgical moving image as the accompanying information by applying an image processing technology disclosed in Japanese Patent Application Laid-Open No. 2011-36371. The accompanying information generated by the accompanying information generation unit 106 can be attached to both a first surgical moving image and a second surgical moving image.

Furthermore, the accompanying information generation unit 106 may tag the generated accompanying information. The tagged accompanying information is displayed in a tag information display field 305 of the node 30 to be described below. Furthermore, the accompanying information generation unit 106 may analyze movement of a person involved in surgery, for example, movement of a nurse from a moving image of an operative field camera by applying an existing image analysis technology, and generate a tag for the movement of the nurse.

The search unit 105 has a function to search for a surgical moving image stored in the storage server 20 or the storage unit 120. Specifically, the search unit 105 searches for an appropriate surgical moving image on the basis of input information using the node 30. The search unit 105 may search for the appropriate surgical moving image on the basis of a surgery ID. A search result by the search unit 105 is output to the node 30 by the output control unit 130.

The output control unit 130 has a function to control information to be output to the node 30 on the basis of authority information. For example, the output control unit 130 controls the surgical moving image to be output to the node 30 and processing executable by the node 30 by referring to the authority information associated with a user ID transmitted from the node 30. Furthermore, the output control unit 130 may have a function to output the search result by the search unit 105 to the node 30.

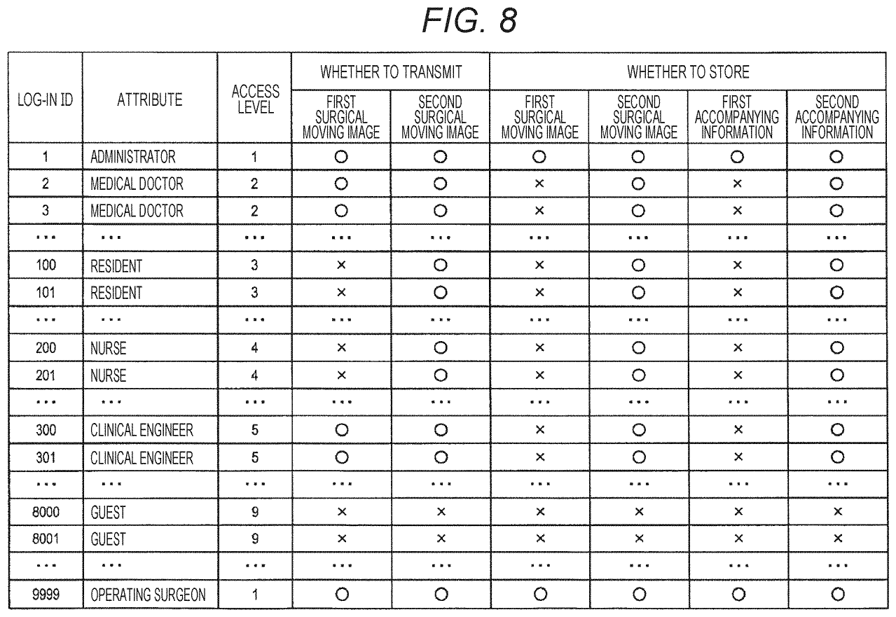

Here, the control performed by the output control unit 130 will be described in detail with reference to FIG. 8. FIG. 8 is a diagram illustrating an example of an authority information list according to the present embodiment. For example, the authority information list includes an attribute, an access level, whether to transmit the surgical moving image, whether to store the surgical moving image and the accompanying information, and the like, associated with the user ID, as illustrated in FIG. 8. The output control unit 130 can control the output to the node 30 according to the access level associated with the user ID. Specifically, the output control unit 130 can control types of surgical moving image and accompanying information that can be output, and types of surgical moving image and accompanying information that can be stored in the node 30, and the like according to the access level.

For example, the output control unit 130 can perform control to transmit the first surgical moving image and the second surgical moving image to the node 30 of the user ID with the access level of “1”. The output control unit 130 can cause the node 30 of the user ID with the access level of “1” to store the first surgical moving image, the second surgical moving image, first accompanying information, and second accompanying information. Furthermore, the output control unit 130 can perform control to transmit the first surgical moving image and the second surgical moving image to the node 30 of the user ID with the access level of “2” or “5”, for example. The output control unit 130 can cause the node 30 with the access level of “2” or “5” not to store the first surgical moving image and the first accompanying information and to store the second surgical moving image and the second accompanying information. Furthermore, for example, the output control unit 130 can perform control not to transmit the first surgical moving image and to transmit the second surgical moving image to the node 30 of the user ID with the access level of “3” or “4”. The output control unit 130 can cause the node 30 with the access level of “3” or “4” not to store the first surgical moving image and the first accompanying information and to store the second surgical moving image and the second accompanying information. Furthermore, the output control unit 130 can perform control not to transmit both the first surgical moving image and the second surgical moving image to the node 30 of the user ID with the access level of “9”, for example. The output control unit 130 can cause the node 30 with the access level of “9” not to store the first surgical moving image, the second surgical moving image, the first accompanying information, and the second accompanying information.

The access level may be set according to the user ID, an attribute, an access place, an access method, the type of the node 30, or the like. For example, for a user with an attribute of an operating surgeon, the access level may be set to be high, and both the first surgical moving image and the second surgical moving image may be made available and many types of accompanying information may be made available. For a user with an attribute of a resident, the access level may be set to be low, and only the second surgical moving image may be made available and limited accompanying information may be made available. Furthermore, for example, the access level may be set to be high for the node 30 that has accessed the information processing device 11 from a network in a hospital, while the access level may be set to be low for the node 30 that has accessed the information processing system 2 via another network. Furthermore, for example, the access level may be set to be high for the node 30 in a case where the node 30 is a PC, while the access level may be set to be low for the node 30 in a case where the node 30 is a mobile terminal.

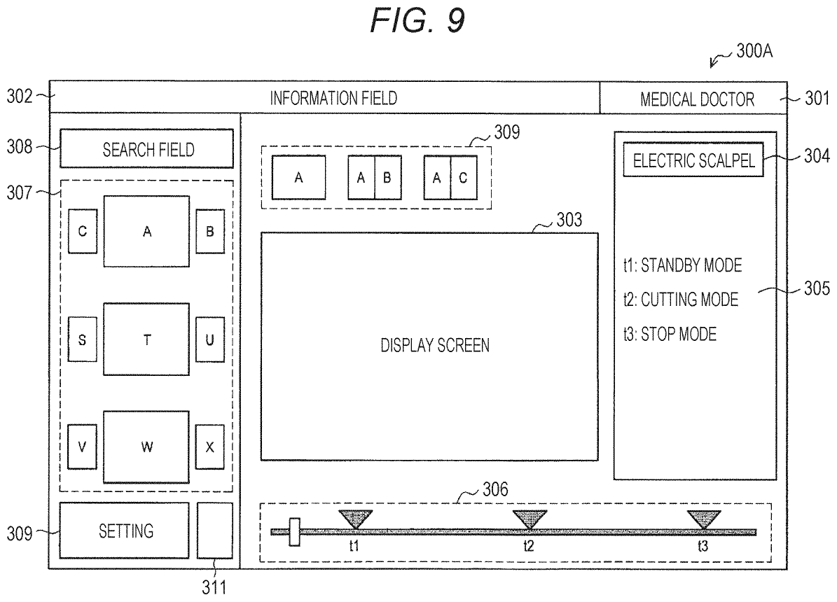

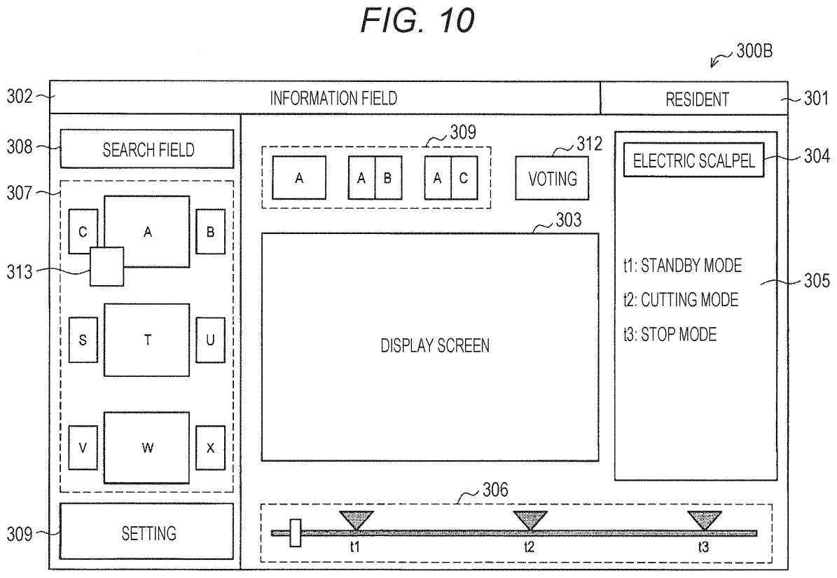

Furthermore, the output control unit 130 may change a display screen output by the node 30 according to the user's attribute or access level. For example, the output control unit 130 may output a first display screen 300A on which the first surgical moving image and the second surgical moving image are switchably displayed, and a second display screen 300B on which only the second surgical moving image is displayed without displaying the first surgical moving image. The first display screen 300A is output to the node 30 to which the determination unit 103 has determined that the first surgical moving image can be transmitted. The second display screen 300B is output to the node 30 to which the determination unit 103 has determined that the first surgical moving image cannot be transmitted but the second surgical moving image can be transmitted. Here, the display screen output by the output control unit 130 to the node 30 will be described in detail with reference to FIGS. 9 and 10. FIG. 9 is an explanatory diagram of the first display screen 300A for displaying the first surgical moving image and the first accompanying information output to the node 30 according to the present embodiment. FIG. 10 is an explanatory diagram of the second display screen 300B for displaying the second surgical moving image and second accompanying information output to the node 30 according to the present embodiment.

First, the first display screen 300A, which is a display screen of the node 30 to which the first surgical moving image and accompanying information are output, will be described with reference to FIG. 9. The first display screen 300A displays an attribute display field 301, an information field 302, a main display area 303, a tag selection field 304, the tag information display field 305, a timeline field 306, a related moving image display field 307, a search field 308, a selection button 309, a surgical moving image conversion button 310, and a setting field 311.

In the attribute display field 301, the attribute associated with the user ID is displayed. In FIG. 9, “medical doctor” as the user's attribute is displayed, for example. In the attribute display field 301, the display item is not limited to the attribute and the user ID or a user's name associated with the user ID may be displayed.

In the information field 302, information regarding a surgical moving image displayed in the main display area 303 is displayed. For example, in the information field 302, information of a patient name, a surgery date, a surgical site, an operating surgeon, surgery conditions, and the like is displayed. The surgery conditions include, for example, laparotomy, endoscopic surgery, presence or absence of complications, and the like.

In the main display area 303, the first surgical moving image is displayed. In the main display area 303, one moving image of the first surgical moving images may be displayed, or a plurality of moving images may be displayed. The moving image displayed in the main display area 303 can be selected by the user using the selection button 309. Furthermore, in the main display area 303, a patient's electronic health record may be displayed.

In the tag selection field 304, a plurality of tags added by the accompanying information generation unit 106 is displayed, and one tag of the plurality of tags is selected by the user. The accompanying information corresponding to the selected tag is displayed in the tag information display field. For example, in a case where the tag of an electric scalpel is selected by the user in the tag selection field 304, information regarding a change in the electric scalpel used in the surgery in which the surgical moving image displayed in the main display area 303 has been acquired is displayed in chronological order in the tag information display field 305. For example, as illustrated in FIG. 9, the electric scalpel being changed from stop mode to standby mode at time t1, the electric scalpel being changed from the standby mode to cutting mode at time t2, and the electric scalpel being changed from the cutting mode to the stop mode at time t3 may be displayed. Furthermore, for example, the accompanying information displayed in the tag information display field 305 may be linked to an icon displayed in the timeline field 306 to be described below. Information displayable in the tag information display field 305 can be accompanying information that changes during surgery, and examples include information regarding each step of the surgery, timing of a change in display of patient's personal information, a change in patient's anesthesia state, occurrence timing of bleeding, a change in the amount of bleeding, occurrence timing of alarm generated by a medical device, a change in a patient state, and the like. Furthermore, when the user selects information regarding each time displayed in the tag information display field 305, a moving image of the selected time may be displayed in the main display area 303.

In the timeline field 306, a timeline of the first surgical moving image displayed in the main display area 303 is displayed. On the timeline, a display indicating a playback position of the moving image displayed in the main display area 303 may be displayed, or an icon displaying time when a predetermined event has occurred may be displayed. The event may be displayed in the tag information display field 305, for example. For example, when the electric scalpel is selected by the user in the tag selection field 304 and the information regarding a change in the electric scalpel is displayed in chronological order in the tag information display field 305, an icon may be displayed at a position corresponding to each time on the timeline. For example, in FIG. 9, icons are displayed at positions corresponding to time t1, time t2, and time t3 on the timeline. When the icon displayed on the timeline is selected by the user, the moving image at the selected time may be displayed in the main display area 303.

In the related moving image display field 307, a surgical moving image related to the surgical moving image displayed in the main display area 303 may be displayed, for example. In the related moving image display field 307, the first surgical moving image in the same surgery as the surgical moving image displayed in the main display area 303 and different from the surgical moving image displayed in the main display area 303 may be displayed, for example. Specifically, in a case of an endoscopic surgery where the first surgical moving image displayed in the main display area 303 is a moving image acquired by an operative field camera, a moving image acquired by an endoscope may be displayed in “A” in the related moving image display field 307. Similarly, for example, a moving image in the same endoscopic surgery and in which a display screen of a peripheral measuring instrument is captured may be displayed in “B” in the related moving image display field 307, and a moving image acquired by a camera that captures a state of a nurse in a line-of-sight direction may be displayed in “C” in the related moving image display field 307.

Furthermore, in the related moving image display field 307, a moving image related to the information displayed in the information field 302 may be displayed, for example. The first surgical moving image of the same site as a surgical site displayed in the information field 302 and of a separately conducted surgery may be displayed in “T” in the related moving image display field 307, for example. Similarly, the first surgical moving image of another surgery conducted by the operating surgeon displayed in the information field 302 may be displayed in “S” in the related moving image display field 307, and the first surgical moving image of surgery close to the surgery conditions displayed in the information field 302 may be displayed in “U” in the related moving image display field 307, for example.

Furthermore, in the related moving image display field 307, the first surgical moving image searched by the user may be displayed, for example. For example, the first surgical moving image having the highest degree of association with a search term input in the search field 308 may be displayed in “W” in the related moving image display field 307. The first surgical moving image having a relatively high degree of association with a search term may be displayed in “V” and “X” in the related moving image display field 307.

In the search field 308, a surgery ID or a search term may be input by the user in order to search for the first surgical moving image desired by the user from among the first surgical moving images stored in the storage server 20. The appropriate first surgical moving image is searched for according to the search term by inputting at least a part of the surgery ID, the patient name, the surgery date, the surgical site, the operating surgeon, the surgery conditions, or the like in the search field 308, for example. The searched first surgical moving image may be displayed in the related moving image display field 307.

The selection button 309 is used by the user to select a surgical moving image and a display format displayed in the main display area 303. The moving image is displayed in the main display area 303 according to the display format displayed on the selection button 309. For example, in FIG. 9, four types of buttons are displayed, and when the button “A” is selected by the user, “A” displayed in the related moving image display field 307 is displayed in the main display area 303. Furthermore, for example, when the button “AB” is selected by the user, “A” and “B” displayed in the related moving image display field 307 are displayed side by side in the main display area 303. By selecting the surgical moving image or the display format with the selection button 309 in this manner, another surgical moving image displayed in the related moving image display field 307 is displayed in the main display area 303. Furthermore, as the predetermined surgical moving image selected with the selection button 309 is selected, the accompanying information displayed in the tag information display field 305 and the timeline displayed in the timeline field 306 are changed to the accompanying information accompanying the selected surgical moving image.

The surgical moving image conversion button 310 is used to convert the surgical moving image displayed on the display screen of the node 30 from the first surgical moving image to the second surgical moving image. The surgical moving image conversion button 310 is displayed on the node 30 having the user ID that can receive the first surgical moving image.

In the setting field 311, the user performs various settings on the display screen of the node 30. In the setting field 311, for example, the order of surgical moving images displayed in the related moving image display field 307 can be changed by a user's instruction, and a surgical moving image with a good surgical result may be displayed above a surgical moving image with a poor surgical result. Furthermore, in the setting field 311, for example, the user sets a font to be used, a character size, a search item, and the like.

Next, the second display screen 300B, which is a display screen of the node 30 to which the second surgical moving image and the second accompanying information are output, will be described with reference to FIG. 10. The second display screen 300B is displayed on the node 30 to which the determination unit 103 has determined that the first surgical moving image cannot be transmitted but the second surgical moving image can be transmitted. Therefore, the second surgical moving image for which protection processing for personal information has been performed is displayed on the second display screen 300B. The second display screen 300B displays an attribute display field 301, an information field 302, a main display area 303, a tag selection field 304, a tag information display field 305, a timeline field 306, a related moving image display field 307, a search field 308, a selection button 309, a setting field 311, a voting button 312, and a reference level display field 313. The attribute display field 301, the information field 302, the timeline field 306, the search field 308, the selection button 309, and the setting field 311 on the second display screen are similar to those displayed on the first display screen, and thus description thereof is omitted here.

In the main display area 303, an electronic health record of the patient may be displayed similarly to the first display screen 300A, but the electronic health record displayed on the second display screen 300B is an electronic health record for which the protection processing for the personal information included in the electronic health record has been performed.

The accompanying information according to the attribute or the access level of the user may be displayed in the tag information display field 305 on the second display screen 300B. For example, in the case where the attribute of the user is a resident, only a progress status of surgery and the change in the electric scalpel may be displayed in the tag information. In the case where the attribute of the user is a clinical engineer, changes in all of medical devices used in the surgery may be displayed. Furthermore, in the case where the attribute of the user is an anesthesiologist, the anesthesia state may be displayed in the tag information display field 305. Furthermore, in the case where the attribute of the user is an anesthesiologist, a facial expression of a patient is important, so information indicating the facial expression of the patient (for example, information of the facial expression after replacement processing has been performed) may be displayed. Furthermore, for example, the accompanying information generation unit 106 analyzes movement of a person involved in the surgery, for example, movement of a nurse from a moving image of an operative field camera, and the tag generated for the movement of the nurse may be displayed in the tag information display field 305. Information regarding surgical tools used in the surgery, specifically, the type of forceps, the type of thread used for suturing, and the like may be displayed in the tag information display field 305, for example. Furthermore, the accompanying information based on billing information may be displayed in the tag information display field 305 on the second display screen 300B. For example, the billing information associated with the accessed user is read from a billing information database including the billing information indicating a billing amount or a billing plan of the user. The accompanying information is displayed in the tag information display field 305 on the second display screen 300B on the basis of table information recorded in advance and in which the billing information and the displayable accompanying information are associated. As a result, the amount of display information based on the billing information can be controlled. Furthermore, information of a position of a blood vessel may be generated by an analysis of a moving image, and a tag displaying the information of the position of the blood vessel may be superimposed and displayed on the moving image.

Furthermore, a tag of “comment” may be registered in advance in the tag selection field 304 as a selection candidate. For example, in a case where the tag of “comment” is selected, a comment input by the user may be displayed in the tag information display field 305 in order of playback time of the moving image. Thereby, information can be exchanged between users who use one surgical moving image at different timings. As a result, the information not included in the accompanying information can be complemented by the user's comment. For example, information regarding a medical device used in surgery, specifically, information regarding a manufacturer, a model, and the like of the medical device, information regarding criteria for selecting a surgical tool used in the surgery, and the like can be exchanged between users.

In the related moving image display field 307, a search result according to the user's attributes or access level may be displayed. For example, in the case where the user is a resident, the resident may be able to search for a surgical moving image by an illness type, an affected part, treatment content, patient's gender, age, or smoking habit, an operating surgeon, or the like.

The reference level display field 313 is arranged corresponding to the second surgical moving image displayed in the related moving image display field 307, and displays a reference level of the surgical moving image. For example, the voting button 312 is a button used by the user when it is determined that the second surgical moving image displayed in the main display area 303 is useful to the user, and in a case where the voting button 312 is pressed by the user, the reference level of the second surgical moving image increases. The second surgical moving image having a high reference level may be displayed above the second surgical moving image having a low reference level in the related moving image display field 307. Furthermore, in the reference level display field 313, supplementary information regarding the corresponding surgical moving image may be displayed. For example, information regarding a publication state of the surgical moving image, specifically, presence or absence of publication, publication purpose, date and time, and the like may be displayed in the reference level display field 313.

Furthermore, the output control unit 130 may cause the second display screen 300B to be displayed differently depending on the attribute or the access level. Furthermore, in the case where the user is a resident, a display of a surgical moving image recommended by a medical doctor may be displayed on the second display screen 300B.

Note that, since the first surgical moving image is not displayed on the second display screen, the surgical moving image conversion button 310 displayed on the first display screen is not displayed.

Furthermore, the output control unit 130 may change the image quality of the surgical moving image displayed on the node 30 depending on the access level.

The surgical system 41 transmits, to the information processing device 11, information regarding an operating state of a medical device used in surgery in addition to the first surgical moving image. The transmitted information regarding an operating state of a medical device may be used as the accompanying information and displayed in the above-described tag information display field 305.

[2.2. Operation]

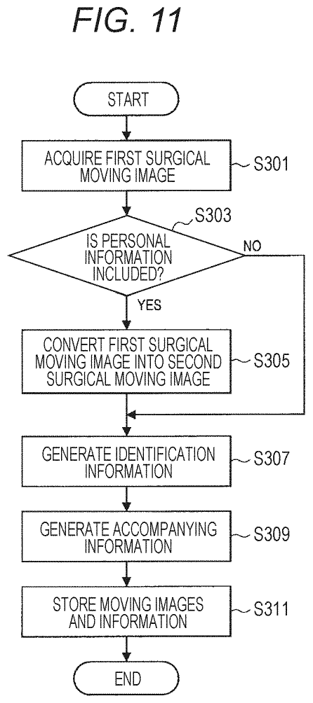

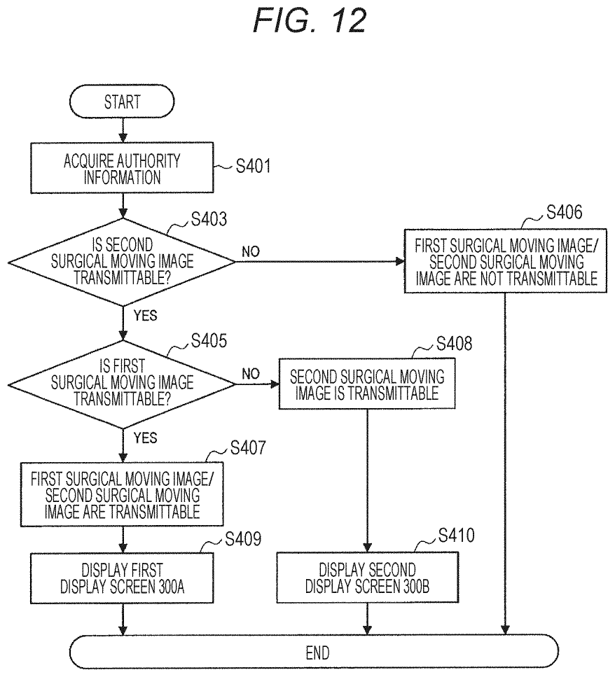

A configuration of the information processing system 2 according to the present embodiment has been described. Next, an operation of the information processing device 11 will be described with reference to FIGS. 11 and 12. FIG. 11 is a flowchart illustrating an operation flow from acquisition to storage of the first surgical moving image by the information processing device 11 according to the present embodiment. FIG. 12 is a flowchart illustrating an operation flow from acquisition to output of the authority information to the node 30 by the information processing device 11 according to the present embodiment.

An operation flow from acquisition of the first surgical moving image to storage of the first surgical moving image, the second surgical moving image, and the accompanying information by the information processing device 10 will be described with reference to FIG. 11. Each of S303, S305, S307, and S311 illustrated in FIG. 11 is similar to S103, S105, S107, and S111 described in the first embodiment, and thus detailed description thereof is omitted here.

First, the information processing device 11 acquires the first surgical moving image from the surgical system 41 provided in the operating room (S301). Specifically, the communication unit 110 receives the first surgical moving image from the surgical system 40 via the network 50 and transmits the first surgical moving image to the information processing unit 100. At this time, the communication unit 110 may receive the information regarding an operating state of a medical device used in the surgery from the surgical system 41. The first surgical moving image and the information regarding an operating state of a medical device may be stored in the storage server 20 or the storage unit 120.

In S309, the accompanying information accompanying the second surgical moving image accompanying the first surgical moving image is generated. Specifically, the accompanying information generation unit 106 generates the accompanying information accompanying any information related to the surgery such as timing at which or time for which the personal information is included in the moving image. For example, the accompanying information generation unit 106 generates, as the accompanying information, information of the type of the surgery, a flow of the surgery, a progress of the surgery, the amount of bleeding, a location of bleeding, occurrence timing of bleeding, timing at which the medical device is used, movement of a nurse, and the like. The accompanying information generation unit 106 generates the accompanying information on the basis of the first surgical moving image, the second surgical moving image, the information regarding an operating state of a medical device transmitted from the surgical system 41, for example.

Next, an operation flow from acquisition to output of the authority information to the node 30 by the information processing device 11 will be described with reference to FIG. 12. Each of S401, S403, S405, S406, S407, and S408 illustrated in FIG. 12 is similar to S201, S203, S205, S206, S207, and S208 described in the first embodiment, and thus detailed description thereof is omitted here.

In a case where the determination unit 103 determines that both the first surgical moving image and the second surgical moving image can be transmitted (S407), the node 30 becomes able to display the first display screen (S409). The user can use, for example, the first surgical moving image, the second surgical moving image, the accompanying information, or a surgical moving image related to the first surgical moving image or the second surgical moving image displayed on the first display screen 300A of the node 30.

Furthermore, in the case where the determination unit 103 determines that the second surgical moving image can be transmitted (S408), the node 30 becomes able to display the second display screen (S410). The user can use the second surgical moving image, the accompanying information, or a surgical moving image related to the second surgical moving image displayed on the second display screen of the node 30.

According to the present embodiment, the node 30 can display the type of surgical moving image and accompanying information according to the access level of the user who uses the surgical moving image. Thereby, use of the surgical moving image becomes possible from a different point of view for one surgery according to the attribute or the purpose of the user while protecting the personal information of the patient displayed in the surgical moving image. As a result, a highly convenient surgical moving image can be provided to the user while protecting the personal information. For example, according to the information processing system of the present embodiment, the surgical moving image in which the personal information of the patient is protected can be used for the use of learning data of learning materials for medical staff and machine learning while storing the unmodified surgical moving image recorded during surgery.

Furthermore, since the surgical moving image recorded during surgery is stored without being modified, the patient can be easily specified by confirming the personal information included in the surgical moving image, as needed.

### 3. Third Embodiment

[3.1. Configuration]

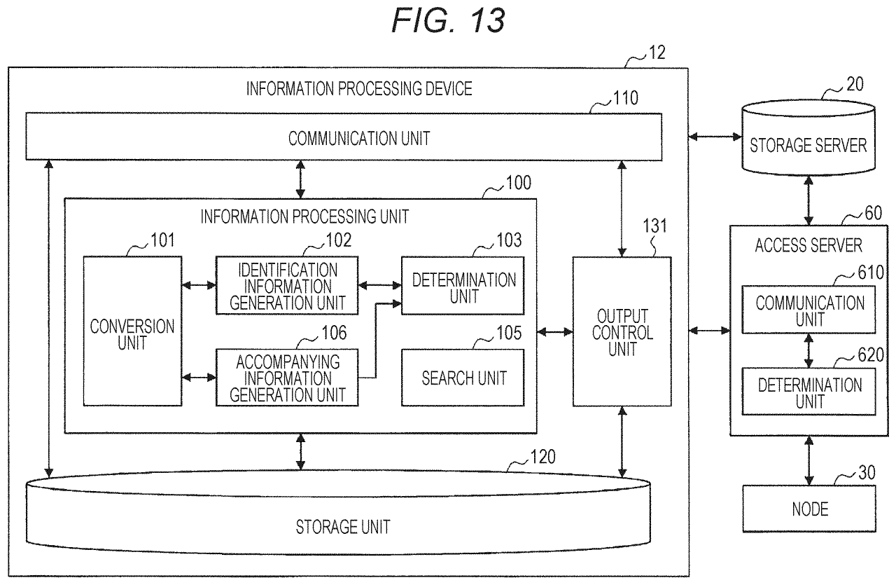

Next, an information processing system 3 according to a third embodiment of the present disclosure will be described with reference to FIG. 13. FIG. 13 is a block diagram illustrating a configuration example of the information processing system 3 according to the present embodiment. The information processing system 3 according to the present embodiment includes an information processing device 12, a storage server 20, a node 30, a surgical system 41 (not illustrated), a network 50 (not illustrated), and an access server 60. In the present embodiment, the storage server 20 and the node 30 applicable to the information processing system 3 are similar to those described in the first embodiment and the surgical system 41 is similar to that described in the second embodiment, and thus detailed description thereof is omitted here.

The information processing device 12 according to the present embodiment includes an information processing unit 100, a communication unit 110, a storage unit 120, and an output control unit 131. The information processing unit 100 includes a conversion unit 101, an identification information generation unit 102, a determination unit 103, an accompanying information generation unit 106, and a search unit 105. In the present embodiment, the conversion unit 101, the identification information generation unit 102, the determination unit 103, the accompanying information generation unit 106, the search unit 105, the communication unit 110, and the storage unit 120 are similar to those described in the second embodiment, and thus detailed description thereof is omitted here.

The output control unit 131 controls a surgical moving image and accompanying information to be output to the node 30. For example, the output control unit 131 controls the surgical moving image and accompanying information corresponding to a determination result of a determination unit 620 included in the access server 60 to be described below.

The node 30 accesses the information processing device 12 and the storage server 20 via the access server 60. The access server 60 includes a communication unit 610 and the determination unit 620.

The communication unit 610 has a function to transmit and receive various types of information to and from the information processing device 10, the storage server 20, and the node 30 via the network 50. Specifically, the communication unit 610 receives a first surgical moving image, a second surgical moving image, accompanying information, and identification information from the surgical system 41 or the storage server 20. Furthermore, the communication unit 610 receives an instruction from the node 30, a user ID, or the like, and transmits the surgical moving image, the accompanying information, and the like to the node 30 according to instruction content from the node 30 or determination by the determination unit 620. Furthermore, the communication unit 610 transmits the first surgical moving image, the second surgical moving image, various types of information generated in the information processing unit 100, such as the identification information, the accompanying information, authority information received from the node 30, and the like to the storage server 20.

The determination unit 620 has a function to determine whether to transmit the first surgical moving image and whether to transmit the second surgical moving image to the node 30 on the basis of the authority information transmitted from the node 30. Specifically, the determination unit 620 determines whether to transmit the surgical moving images on the basis of the user ID transmitted from the node 30 and an authority information list illustrated in FIG. 3, for example.

[3.2. Operation]

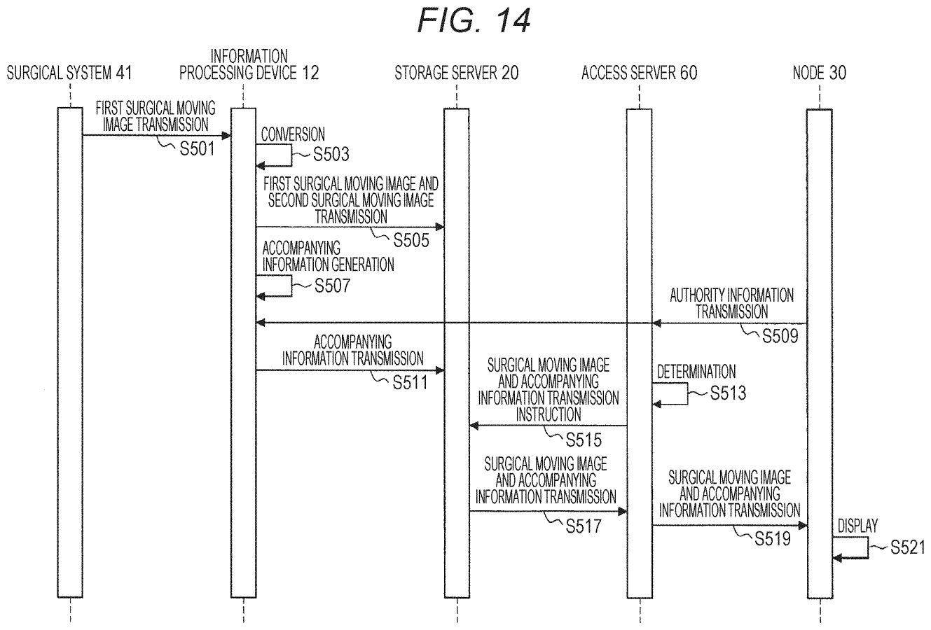

Next, an operation of the information processing system 3 will be described with reference to FIG. 14. FIG. 14 is a sequence diagram illustrating an operation flow from acquisition to storage of the first surgical moving image by the information processing system 3 according to the present embodiment.

The surgical system 41 transmits, to the information processing device 12, the first surgical moving image and information regarding an operating state of a medical device used in surgery to the information processing device 10 (S501). In a case where personal information is included in the first surgical moving image, the information processing device 12 converts the first surgical moving image into the second surgical moving image using the conversion unit 101 (S503). The first surgical moving image and the second surgical moving image are transmitted to the storage server 20 (S505). Furthermore, the information processing device 12 generates the accompanying information accompanying the first surgical moving image and the second surgical moving image using the accompanying information generation unit 104 (S507). The node 30 transmits the authority information to the information processing device 12 and the access server 60 (S509). The information processing device 12 selects the accompanying information transmittable to the node 30 of the user who has the authority on the basis of the transmitted authority information, and transmits the accompanying information to the storage server 20 (S511). The access server 60 determines, in the determination unit 620, whether to transmit the first surgical moving image and whether to transmit the second surgical moving image to the node 30 on the basis of the authority information transmitted from the node 30 (S513). The access server 60 transmits a transmission instruction of the surgical moving image and the accompanying information to the storage server 20 on the basis of a determination result (S515). The storage server 20 transmits the corresponding surgical moving image and accompanying information to the access server 60 on the basis of the transmission instruction transmitted from the access server 60 (S517). Next, the access server 60 transmits the received surgical moving image and accompanying information to the node 30 (S519). Then, the node 30 displays the received surgical moving image and accompanying information (S521). For example, the node 30 displays a first display screen when receiving the first surgical moving image, and displays a second display screen when receiving the second surgical moving image without receiving the first surgical moving image.

According to the information processing system of the present embodiment, the information processing device 12 transmits the accompanying information corresponding to present authority information to the storage server 20. Therefore, transmission of accompanying information without authority to the node 30 can be prevented. Furthermore, transmission of the surgical moving image to the node 30 is determined by the determination unit 620 included in the access server 60 that is physically distant from the information processing device 12 that generates the accompanying information. Therefore, a possibility of leakage of information can be further reduced.

[3.3. Modification]

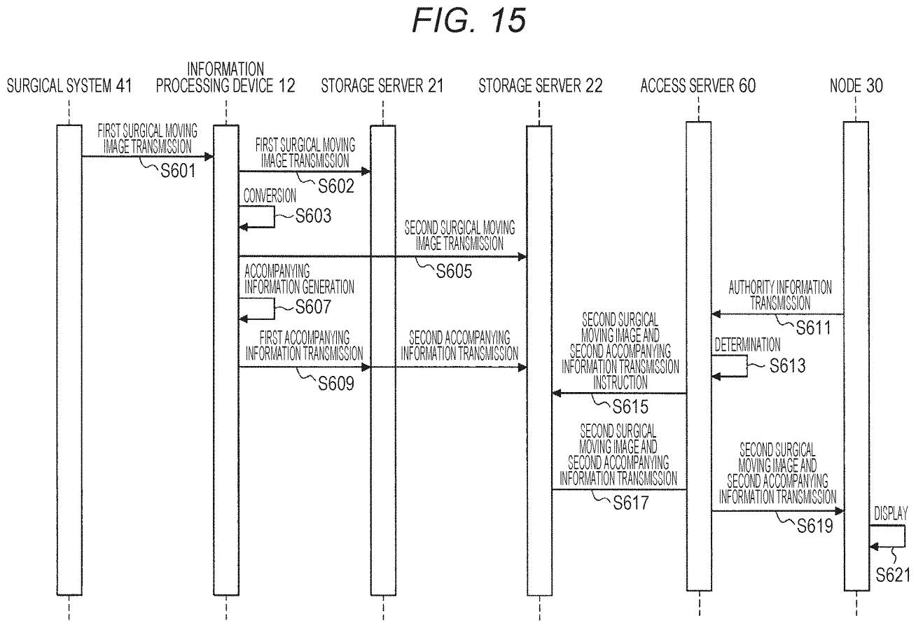

In the above embodiment, a case in which one storage server 20 is provided has been described, but the information processing system 3 according to the present embodiment may include a plurality of storage servers. For example, in a case where two storage servers are provided, one storage server 21 may store the first surgical moving image and the other storage server 22 may store the second surgical moving image. An operation flow from acquisition to storage of the second surgical moving image in a case where the first surgical moving image and the second surgical moving image are respectively stored in different storage servers will be described with reference to FIG. 15. FIG. 15 is a sequence diagram illustrating an operation flow from acquisition to storage of the second surgical moving image in the case where the first surgical moving image and the second surgical moving image are respectively stored in different storage servers. Note that S601, S603, and S613 illustrated in FIG. 15 are similar to the above-described S501, S503, and S513, respectively, and thus description thereof is omitted here. The storage server 21 corresponds to a first storage server according to the present disclosure, and the storage server 22 corresponds to a second storage server according to the present disclosure.

The first surgical moving image transmitted to the information processing device 12 is transmitted to the storage server 21 (S602). After the first surgical moving image is converted into the second surgical moving image by the information processing device 12 (S603), the information processing device 12 transmits the second surgical moving image to the storage server 22 (S605). Next, the accompanying information generation unit 106 included in the information processing device 12 generates first accompanying information and second accompanying information (S607). Next, the information processing device 12 transmits the first accompanying information to the storage server 21 and the second accompanying information to the storage server 22 (S609).

The node 30 transmits the authority information to the access server 60 (S611). In S613, in a case where the determination unit 620 determines that the first surgical moving image cannot be transmitted and the second surgical moving image can be transmitted to the node 30, the transmission instruction of the second surgical moving image and the second accompanying information is transmitted to the storage server 22 (S615). The storage server 22 transmits the second surgical moving image and the second accompanying information to the access server 60 on the basis of the transmission instruction transmitted from the access server 60 (S617). Next, the access server 60 transmits the received second surgical moving image and second accompanying information to the node 30 (S619). Then, the node 30 displays the received second surgical moving image and second accompanying information (S621).

In the above description, a case in which the access server 60 receives the second surgical moving image and the second accompanying information stored in the storage server 22 and transmits the second surgical moving image and the second accompanying information to the node 30 has been described. However, the embodiment is not limited to the example, and the transmission instruction of the surgical moving image and the accompanying information may be transmitted to at least either the storage server 21 or the storage server 22 on the basis of the determination result by the access server 60. For example, in a case where the determination unit 620 determines that the first surgical moving image and the second surgical moving image can be transmitted, the transmission instruction of the surgical moving image and the accompanying information may be transmitted to the storage server 21 and the storage server 22. Thereafter, the storage server 21 may transmit the first surgical moving image and the first accompanying information to the access server 60, and the storage server 22 may transmit the second surgical moving image and the second accompanying information to the access server 60.

With the plurality of storage servers, the first surgical moving image and the second surgical moving image can be stored in the different storage servers. As a result, security is further enhanced and leakage of the personal information can be prevented. Furthermore, only a node having an access level equal to or higher than a prescribed level can be made connectable to the storage server that stores the first surgical moving image. For example, the determination unit 620 determines an access location from the access level, and blocks the access to the storage server that stores the first surgical moving image in a case where the access is from an external network other than an in-hospital network. Thereby, the security can be further strengthened.

Note that a server in the hospital may be set as the storage server 21 that records the first surgical moving image including the personal information, and a cloud server (a server outside the hospital set on a network by cloud computing) may be set as the storage server 22 that records the second surgical moving image not including the personal information. At this time, it is favorable that an access via an access server provided in the hospital is made accessible to the storage server 21, and an access via an access server provided on the cloud is made accessible only to the storage server 22. Thereby, the security can be enhanced by blocking the access to the storage server that stores the first surgical moving image, and convenience can be improved by enabling an access by the cloud computing. Furthermore, at this time, some functions of the information processing device 12 described in the above embodiment may be provided in the cloud server. For example, the information processing device in the hospital records the surgical moving image in the storage server 21 in the hospital as the first surgical moving image associated with the first identification information, and transmits the surgical moving image to the cloud server. The cloud server performs protection processing of protecting the personal information for the surgical moving image, generates a second surgical moving image associated with the second identification information, and records the second surgical moving image on the cloud server. Thereby, the protection processing for the personal information can be performed by calculation resources of the cloud server, and thus adjustment of the calculation resources is facilitated. At this time, the first surgical moving image is recorded on the cloud server, and a private key and a public key are generated, and only the user having the private key may be made accessible to the first surgical moving image. Furthermore, the information processing device 12 may record patient's pre-surgery consent information (for example, information indicating consent to record a surgical moving image or publish the surgical moving image after the personal information is protected) in association with the surgical moving image. Moreover, a determination unit that determines the presence of the protection processing for the personal information or the record in the storage server 22 may be included on the basis of whether or not the consent information is associated with the surgical moving image.

### 4. Hardware Configuration

The embodiments according to the present disclosure have been described. The above-described image processing is implemented by cooperation of software and hardware of the above-described information processing system.

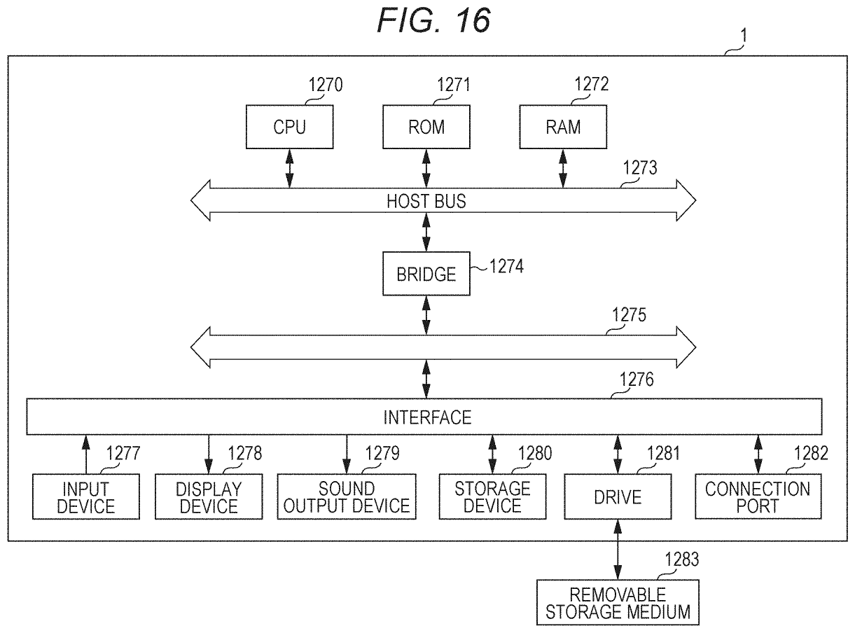

Next, a hardware configuration example of the information processing system 1 according to an embodiment of the present disclosure will be described. FIG. 16 is a block diagram illustrating a hardware configuration example of the information processing system 1 according to the embodiment of the present disclosure. Referring to FIG. 16, the information processing system 1 includes, for example, a CPU 1270, a ROM 1271, a RAM 1272, a host bus 1273, a bridge 1274, an external bus 1275, an interface 1276, an input device 1277, a display device 1278, a sound output device 1279, a storage device 1280, a drive 1281, a connection port 1282, and a removable storage medium 1283. Note that the hardware configuration illustrated here is an example, and some of the configuration elements may be omitted. Furthermore, a configuration element other than the configuration elements illustrated here may be further included.

The CPU 1270 functions as, for example, an arithmetic processing unit or a control unit, and controls the overall operation or part of the operation of the configuration elements on the basis of various programs recorded in the ROM 1271, the RAM 1272, the storage device 1280, or the removable storage medium 1283. The CPU 1270 implements the functions of, for example, the information processing unit 100, the output control unit 130, the output control unit 131, a determination unit 230, and the determination unit 620 in cooperation with software.

The ROM 1271 is a means for storing a program read by the CPU 1270, data used for calculation, and the like. The RAM 1272 temporarily or permanently stores, for example, a program read by the CPU 1270, various parameters that change as appropriate when the program is executed, and the like.

The CPU 1270, the ROM 1271, and the RAM 1272 are connected to one another via, for example, the host bus 1273 capable of high-speed data transmission. Meanwhile, the host bus 1273 is connected to the external bus 876 having a relatively low data transmission speed via the bridge 1274, for example. Furthermore, the external bus 1275 is connected to various configuration elements via the interface 1276.

As the input device 1277, for example, a mouse, a keyboard, a touch panel, a button, a switch, a lever, and the like can be used. Moreover, as the input device 1277, a remote controller capable of transmitting a control signal using infrared rays or other radio waves may be used. Furthermore, the input device 1277 includes a sound input device such as a microphone. The input device 1277 is applied to the node 30, for example. Furthermore, in the surgical system to which the technology of the present disclosure is applicable, a foot switch 250 is favorably used as the input device 1277.

The display device 1278 is, for example, a display device such as a cathode ray tube (CRT), an LCD, or an organic EL, a printer, or the like, and the sound output device 1279 is, for example, an audio output device such as a speaker or headphones. The display device 1278 and the sound output device 1279 are both devices capable of visually or audibly notifying the user of acquired information and are used for the node 30, for example. Furthermore, for example, in a case where a display device is used as the display device 1278, the display device 1278 displays at least one of the above-described first display screen 300A or second display screen 300B.

The storage device 1280 is a device for storing various data. As the storage device 1280, for example, a magnetic storage device such as a hard disk drive (HDD), a semiconductor storage device, an optical storage device, a magneto-optical storage device, or the like is used. The storage device 1280 implements, for example, a function as the storage unit 120 in cooperation with software, and implements one function of the storage server 20, the storage server 21, the storage server 22, or the storage server 23.

The drive 1281 is a device that reads information recorded on the removable storage medium 1283 such as a magnetic disk, an optical disk, a magneto-optical disk, or a semiconductor memory, or writes information to the removable storage medium 1283, for example.

The removable storage medium 1283 is, for example, a DVD medium, a Blu-ray (registered trademark) medium, an HD DVD medium, various semiconductor storage media, or the like. Of course, the removable storage medium 1283 may be, for example, an IC card on which a non-contact IC chip is mounted, an electronic device, or the like.

The connection port 1282 is a port for connecting an external connection device 902 such as a universal serial bus (USB) port, an IEEE1394 port, a small computer system interface (SCSI), an RS-232C port, or an optical audio terminal, for example.

### 5. Conclusion

Although the favorable embodiments of the present disclosure have been described in detail with reference to the accompanying drawings, the technical scope of the present disclosure is not limited to such examples. It is obvious that persons having ordinary knowledge in the technical field of the present disclosure can conceive various modifications or alterations within the scope of the technical idea described in the claims, and the modifications and alterations are naturally understood to belong to the technical scope of the present disclosure.

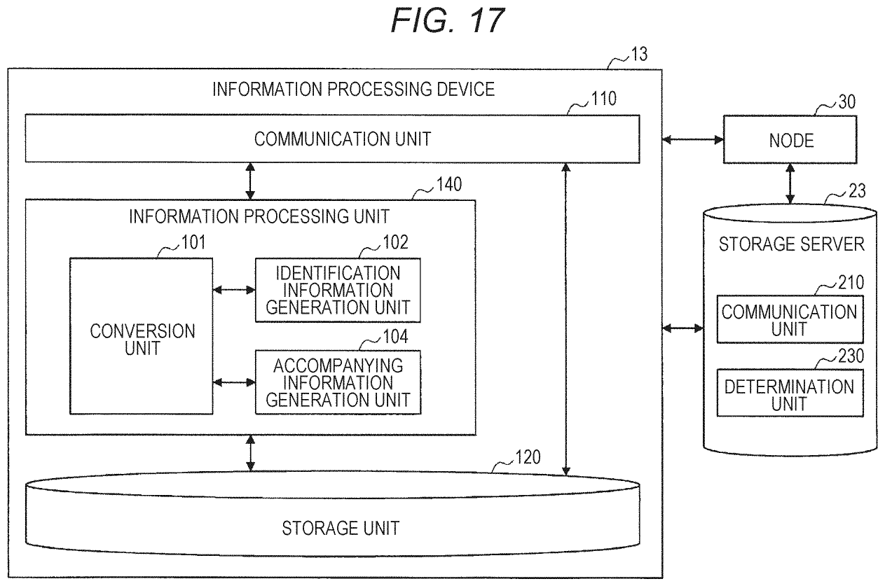

For example, in the above embodiments, the determination unit is provided in the information processing device or the access server, but the present technology is not limited to this example. For example, as illustrated in FIG. 17, the storage server 23 may include the determination unit 103 or the determination unit 230 corresponding to the determination unit 620.

Furthermore, the effects described in the present specification are merely illustrative or exemplary and are not restrictive. That is, the technology according to the present disclosure can exhibit other effects obvious to those skilled in the art from the description of the present specification together with or in place of the above-described effects.

Note that following configurations also belong to the technical scope of the present disclosure.

(1)

An information processing system including:

an acquisition unit configured to acquire a first surgical record that is a record of information regarding an operation of a medical device used in surgery in a hospital;

a conversion unit configured to perform protection processing for personal information of a patient included in the first surgical record to convert the first surgical record into a second surgical record;

an identification information generation unit configured to generate first identification information associated with the first surgical record and second identification information associated with the second surgical record; and

a determination unit configured to determine whether or not the user is accessible to either the first surgical record associated with the first identification information or the second surgical record associated with the second identification information on the basis of authority information regarding an authority of the user determined on the basis of an attribute in the hospital or information of the surgery.

(2)

The information processing system according to (1), in which the first surgical record and the second surgical record include a record by a moving image.

(3)

The information processing system according to (1) or (2), in which, in a case where an access authority to the first surgical record is included in the authority information, the determination unit determines that the first surgical record associated with the first identification information is transmittable to a node used by the user.

(4)

The information processing system according to any one of (1) to (3), in which, further including: an output control unit configured to output the first surgical record or the second surgical record to the node.

(5)

The information processing system according to any one of (1) to (4), further including: an accompanying information generation unit configured to generate accompanying information regarding the personal information accompanying the first surgical record or the second surgical record.

(6)

The information processing system according to (5), in which the accompanying information includes information regarding the surgery.

(7)

The information processing system according to (6), further including:

an output control unit configured to output the first surgical record or the second surgical record to a node used by the user, in which

the output control unit is able to output the accompanying information to the node on the basis of the authority information of the user.

(8)

The information processing system according to (6), further including:

an output control unit configured to output the first surgical record or the second surgical record to a node used by the user, in which

the output control unit outputs the accompanying information together with the first surgical record to the node in a case where the determination unit determines that the first surgical record associated with the first identification information is to be transmitted to the node.

(9)

The information processing system according to any one of (1) to (8), in which

the first surgical record includes sound information, and

the conversion unit performs the protection processing for the personal information of the patient included in the sound information to convert the first surgical record into the second surgical record.

(10)

The information processing system according to any one of (1) to (9), further including:

a storage unit configured to store the first surgical record, the second surgical record, the first identification information, and the second identification information.

(11)

The information processing system according to (10), in which the storage unit is configured by a first storage server that stores the first surgical record and the first identification information and a second storage server that stores the second surgical record and the second identification information.

(12)

The information processing system according to (11), in which

the authority information includes information regarding whether or not a node used by the user is connected to a network in the hospital, and

in a case where the node is not connected to the network in the hospital, the determination unit blocks an access to the first storage server.

(13)

The information processing system according to any one of (1) to (12), in which

the conversion unit performs replacement processing of replacing a face of the patient with a face of another human.

(14)

The information processing system according to (7), in which the output control unit selects the accompanying information to be output on the basis of the authority information of the user.

(15)

The information processing system according to (7), in which the output control unit selects the accompanying information to be output on the basis of the authority information of the user and billing information of the user.

(16)

An information processing device including:

a determination unit configured to determine which of a first surgical record associated with first identification information and a second surgical record associated with second identification information is to be transmitted to a node used by a user on the basis of authority information regarding an authority of the user, in which

the first surgical record is a record of information regarding an operation of a medical device used in surgery, and

the second surgical record is a record converted from the first surgical record when protection processing is performed for personal information of a patient included in the first surgical record.

(17)

An information processing method including:

by a processor,

acquiring a first surgical record that is a record of information regarding an operation of a medical device used in surgery;

performing protection processing for personal information of a patient included in the first surgical record to convert the first surgical record into a second surgical record that is a surgical record from which the patient is unspecifiable;

generating first identification information associated with the first surgical record and second identification information associated with the second surgical record; and

determining which of the first surgical record associated with the first identification information and the second surgical record associated with the second identification information is to be transmitted to a node used by a user on the basis of authority information regarding an authority of the user.

### REFERENCE SIGNS LIST

1, 2, 3 Information processing system
10, 11, 12, 13 Information processing device
20, 21, 22, 23 Storage server
30 Node
40, 41 Surgical system
50 Network
60 Access server
100, 140 Information processing unit
101 Conversion unit
102 Identification information generation unit
103, 230, 620 Determination unit
104, 106 Accompanying information generation unit
105 Search unit
110, 610 Communication unit
120 Storage unit
130, 131 Output control unit
300A First display screen
300B Second display screen

1. An information processing system comprising:
an acquisition unit configured to acquire a first surgical record that is a record of information regarding an operation of a medical device used in surgery in a hospital;
a conversion unit configured to perform protection processing for personal information of a patient included in the first surgical record to convert the first surgical record into a second surgical record;
an identification information generation unit configured to generate first identification information associated with the first surgical record and second identification information associated with the second surgical record; and
a determination unit configured to determine whether or not the user is accessible to either the first surgical record associated with the first identification information or the second surgical record associated with the second identification information on a basis of authority information regarding an authority of the user determined on a basis of an attribute in the hospital or information of the surgery.

2. The information processing system according to claim 1, wherein the first surgical record and the second surgical record include a record by a moving image.

3. The information processing system according to claim 1, wherein, in a case where an access authority to the first surgical record is included in the authority information, the determination unit determines that the first surgical record associated with the first identification information is transmittable to a node used by the user.

4. The information processing system according to claim 3, further comprising:
an output control unit configured to output the first surgical record or the second surgical record to the node.

5. The information processing system according to claim 1, further comprising:
an accompanying information generation unit configured to generate accompanying information regarding the personal information accompanying the first surgical record or the second surgical record.

6. The information processing system according to claim 5, wherein the accompanying information includes information regarding the surgery.

7. The information processing system according to claim 6, further comprising:
an output control unit configured to output the first surgical record or the second surgical record to a node used by the user, wherein
the output control unit is able to output the accompanying information to the node on a basis of the authority information of the user.

8. The information processing system according to claim 6, further comprising:
an output control unit configured to output the first surgical record or the second surgical record to a node used by the user, wherein
the output control unit outputs the accompanying information together with the first surgical record to the node in a case where the determination unit determines that the first surgical record associated with the first identification information is to be transmitted to the node.

9. The information processing system according to claim 1, wherein
the first surgical record includes sound information, and
the conversion unit performs the protection processing for the personal information of the patient included in the sound information to convert the first surgical record into the second surgical record.

10. The information processing system according to claim 1, further comprising:
a storage unit configured to store the first surgical record, the second surgical record, the first identification information, and the second identification information.

11. The information processing system according to claim 10, wherein the storage unit is configured by a first storage server that stores the first surgical record and the first identification information and a second storage server that stores the second surgical record and the second identification information.

12. The information processing system according to claim 11, wherein
the authority information includes information regarding whether or not a node used by the user is connected to a network in the hospital, and
in a case where the node is not connected to the network in the hospital, the determination unit blocks an access to the first storage server.

13. The information processing system according to claim 1, wherein
the conversion unit performs replacement processing of replacing a face of the patient with a face of another human.

14. The information processing system according to claim 7, wherein the output control unit selects the accompanying information to be output on a basis of the authority information of the user.

15. The information processing system according to claim 7, wherein the output control unit selects the accompanying information to be output on a basis of the authority information of the user and billing information of the user.

16. An information processing device comprising:
a determination unit configured to determine which of a first surgical record associated with first identification information and a second surgical record associated with second identification information is to be transmitted to a node used by a user on a basis of authority information regarding an authority of the user, wherein
the first surgical record is a record of information regarding an operation of a medical device used in surgery, and
the second surgical record is a record converted from the first surgical record when protection processing is performed for personal information of a patient included in the first surgical record.

17. An information processing method comprising:
by a processor,
acquiring a first surgical record that is a record of information regarding an operation of a medical device used in surgery;
performing protection processing for personal information of a patient included in the first surgical record to convert the first surgical record into a second surgical record that is a surgical record from which the patient is unspecifiable;
generating first identification information associated with the first surgical record and second identification information associated with the second surgical record; and
determining which of the first surgical record associated with the first identification information and the second surgical record associated with the second identification information is to be transmitted to a node used by a user on a basis of authority information regarding an authority of the user.
